(19) United States
(12) Patent Application Publication
(10) Pub. No.: US 2003/0187034 A1
(43) Pub. Date: Oct. 2, 2003

(54) Composition containing at least one aryl oxime and at least one active substance for treating acne and the use thereof

(76) Inventors: Joachim Bunger, Darmstadt (DE); Hansjurgen Driller, Umstadt (DE); Michael Schwarz, Weiterstadt (DE); Wolfgang Wohlrab, Halle (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/296,058

(22) PCT Filed: May 8, 2001
(86) PCT No.: PCT/EP01/05221

(30) Foreign Application Priority Data
May 24, 2000 (DE) 100255531

(51) Int. Cl. A61K 31/44; A61K 31/40; A61K 31/15

(52) U.S. Cl. 514/357; 514/408; 514/540; 514/640

(57) ABSTRACT

The present invention relates to a composition, comprising

(a) at least one aryl oxime of Formula (I) and

(b) at least one active agent for the treatment of acne

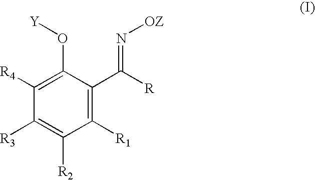

wherein:

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl.

Moreover, the present invention relates to the use of this composition as well as the use of the aforementioned aryl oxime of Formula (I).

The present invention relates to a composition, comprising at least one aryl oxime and at least one active agent for the treatment of acne, as well as the use of this composition. Furthermore, the present invention relates to the use of at least one aryl oxime for prophylaxis and/or for the treatment of skin reddening and/or inflammation caused by acne.

Acne is a very widespread disease of the sebaceous follicle system caused mainly by hormonal, microbial, genetic and/or immunological influences. There are different forms and causes of acne. The forms of acne include endogeneously induced acne, medicamentously induced acne and acne induced by external causes. The most common acne is acne vulgaris, a form of acne induced mainly by endogeneous factors and which is puberty dependent. Acne vulgaris affects regions of the body with a particular type of large sebaceous gland follicles, i.e. mainly the face, the upper back and the upper front line of sweat. In this order, white comedos, black comedos, papulas, pustules, knots and abscessa occur. The abscessa may form channels under the skin which in turn discharge the puss externally through the skin as fistulas. Consequently, it is differentiated between a comedo acne, a papular, papulo-pustular, cystic and induced acne. Some authors regard the acne conglobata as induced acne.

The commedo, commonly known as a blackhead, is the first symptom of acne. It is a plug of horny tissue made of layered horny lamallae mixed with sebaceous matter. In the center it also contains micro-organisms such as propionibacterium acnes, a normal inhabitant of the ducts of the sebaceous glands, and yeast. Early comedos are whitish whereas in older comedos melanin is collected at the tip and therefore the head of the comedos is black. It is differentiated between older open, mostly black, comedos which can be picked easily from the follicle orifice and the immature closed comedos with a narrow follicle construction. With these comedos there is the danger that upon pressure the hair follicle perforates and horny tissue, sebaceous matter and bacteria are pressed into the dermis and cause an inflammation there.

Acne vulgaris is caused by androgenic hormones which stimulate the sebaceous glands; however, other components must also be present. Due to an excess homification in the orifice of the follicle, i.e. the formation of the comedo, the deposition of sebaceous matter from the sebaceous glands and the follicle is impeded or blocked. In the trapped sebaceous matter, micro-organisms reproduce. Therefore, perifollicular inflammation, formation of abscessa with destruction of the sebaceous gland and the advance of free fatty acids into the dermis occur. This causes in turn a further liquifaction of the tissue and a foreign-body response. The skin is undermined and the abscess perforates to the surface.

It is therefore the object of the present invention to provide a composition with improved properties which is suitable for the prophylaxis and/or treatment of acne and which can simultaneously prevent or treat skin reddening and/or inflammation caused by acne and/or acne therapy as an unwanted side effect.

This object is achieved with a composition, comprising

(a) at least one aryl oxime of Formula (I) and

(b) at least one active agent for the treatment of acne

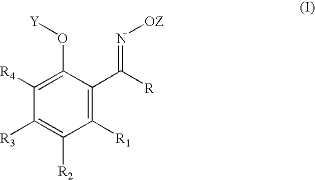

wherein:

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl.

The present invention further relates to the use of said composition for the prophylaxis and/or treatment of acne or for the cosmetic care of the skin.

Furthermore, the present invention relates to the use of at least one aryl oxime of Formula (I)

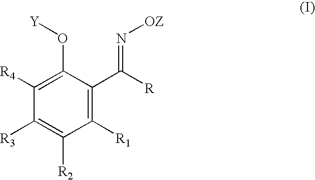

wherein:

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems;

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,

for the prophylaxis and/or treatment of skin reddening and/or inflammation caused by acne and/or acne therapy.

Surprisingly, it was found that by means of the preparation according to the present invention, not only the respective therapeutic effect of the employed active agent is observed, but the inflammation response of the skin is reduced or prevented and the irritative erythema responses observed as unwanted side-effects during acne therapy, e.g. after benzoyl peroxide or trentinoin, can be prevented or minimised.

The preparation of the present invention contains as component (a), at least one aryl oxime of Formula (I)

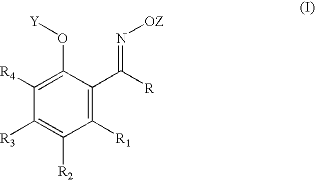

wherein:

Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;

R represents C₁₋₁₈ alkyl, C₂₋₁₈ alkenyl, C₃₋₈ cycloalkyl, aryl, aralkyl, heteroaryl, heteroaralkyl or condensed systems,

R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl.

Alkyl, alkenyl, carboxy alkyl, carboxy alkenyl, alkanoyl, cycloalkyl, alkoxy, aryl, aryloxy, and aralkyl can be unsubstituted or substituted. Suitable substituents of these groups are preferably alkyl, alkoxy, alkenyl, aryl, aryloxy, aralkyl, heteroaryl, heteroaralkyl, hydroxy, carboxy, carboxy alkyl, dialkyl amine, sulfonyl and combinations thereof.

Alkyl is respectively straight chain or branched alkyl and is therefore preferably methyl, ethyl, propyl, isopropyl, butyl, isobutyl, t-butyl, pentyl, hexyl, heptyl, octyl, nonyl, decyl, undecyl, dodecyl, tridecyl, tetradecyl, pentadecyl, hexadecyl, heptadecyl and octadecyl.

Alkenyl means that in the specified alkylene moiety one or more double bonds may be present.

Aryl is an aromatic C₆₋₂₀ hydrocarbon residue and is prefereably phenyl.

Aralkyl is an alkyl group substituted with aryl and has preferably the meaning of benzyl or phenethyl.

Cycloalkyl is a cyclic alkyl group and is preferably cyclopropyl, cyclobutyl, cyclopentyl, cyclohexyl, cycloheptyl or cyclooctyl.

Heteroaryl is an aromatic ring with heteroatoms, preferably a nitrogen-containing ring, such as pyridyl or pyrimidyl.

Heteroaralkyl means a alkyl group substituted with heteroaryl and is preferably pyridyl methyl and pyrimidyl methyl.

Suitable condensed systems are preferably the residues naphthyl, benzofuryl, quinolinyl, indolyl or cinnolinyl.

Dialkyl amine means NR₅R₆ wherein R₅ and R₆ may be the same or different, and C₁₋₁₂ alkyl.

Z and Y are preferably independently from each other a hydrogen atom, a Cl-₆ alkyl group which can have at least one substituent selected from —OH, —COOH, —SO₃H or NR₅R₆, an alkanoyl group represented by —C(O)R₇ wherein R₇ is a C₁₋₆ alkyl group which may have at least one substituent selected from —OH, —COOH or —SO₃H, or a CONHR₈ group wherein R₈ is a C₆₋₂₀ aryl group. Particulary preferably Z and Y are independently from each other a hydrogen atom, —(CH₂)₁₋₆COOH, —CH₂CH(OH)CH₂OH, —(CH₂)₁₋₆SO₃H, —(CH₂)₁₋₆NR₅R₆ or C(O)(CH₂)₁₋₆COOH.

Substituent R is preferably a C₁₋₁₂ alkyl group, particularly preferred are C₁₋₅ and C₁₁ alkyl groups.

Substituent R₁ is preferably a hydrogen atom or a chlorine atom.

Substituent R₂ is preferably a hydrogen atom or a chlorine atom or a C₁₋₆ alkyl group. Particularly preferred are a hydrogen atom, a chlorine atom and a methyl group.

Substituent R₃ is preferably a hydrogen atom or a C₁₋₆ alkyl group, a C₁₋₆ alkoxy group, a O-cyclohexyl group or a benzyl group.

Substituent R₄ is preferably a hydrogen atom or a chlorine atom.

R₁, R₂, R₃ and R₄ may be preferably substituted, if possible, with —OH, —COOH, —SO₃H or —NR₅R₆ to increase, e.g. the water solubility.

Preferred examples of component (a) include:

4-methyl-2-hydroxy-caprophenone oxime, 5-methyl-2-hydroxy-caprophenone oxime, 5-methyl-2-hydroxy-caprophenone (N-phenylcarbamoyl) oxime, 5-methyl-2-hydroxy laurophenone oxime (2-hydroxy-5-methyl-laurophenone oxime), 3-chloro-2-hydroxy-caprophenone oxime, 4-pentoxy-2-hydroxy-acetophenone oxime, 4-decyloxy-2-hydroxy-acetophenone oxime, 4-benzyloxy-2-hydroxy-acetophenone oxime, 4-decyloxy-2-hydroxy-propiophenone oxime, 4-butoxy-5-n-hexyl-2-hydoxy-acetophenone oxime, 4-pentoxy-2-hydroxy-caprophenone oxime, 4-decyloxy-2-hydroxy-caprophenone oxime, 4-octyloxy-2-hydroxy-laurophenone oxime, 4-cyclohexyl-oxy-2-hydroxy-propiophenone oxime, 5-chloro-2-hydroxycaprophenone oxime, 3-chloro-2-hydroxy-laurophenone oxime, 5-chloro-2-hydroxy-laurophenone oxime, 4-butoxy-2-hydroxy-acetophenone oxime, 4-dodecyloxy-2-hydroxy-propiophenone oxime, 4-hexadecyloxy-2-hdroxy-acetophenone oxime, 4-octadecyloxy-2-hydroxy-acetophenone oxime, 4-decyloxy-2-hydroxy-laurophenone oxime, as well as the following oxime derivatives of 2-hydroxy-5-methyl-laurophenone oxime:

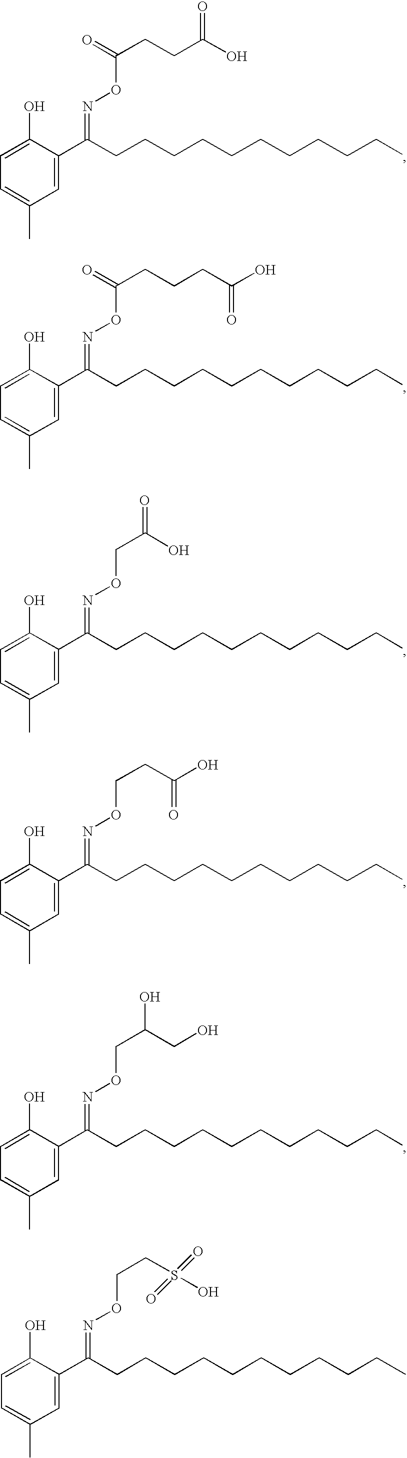

Particularly preferred are 2-hydroxy-5-methyl laurophenone oxime as well as its oxime derivatives mentioned before.

Component (a) is present in the composition according to the present invention in a sufficient amount to be suitable for a cosmetic or dermatological use, in particular for the prophylaxis and/or treatment of skin reddening and/or inflammations caused by acne. Typically, component (a) is present in the composition of the present invention in an amount of 0.005 to 5 wt %, preferably 0.02 to 2 wt %, more preferably 0.05 to 1.5 wt %.

The composition of the present invention comprises as component (b) at least one active agent for the treatment of acne. Typically, component (b) is selected from the group consisting of chemotherapeutics, antibiotics, antiandrogenes, retinoids, benzoyl peroxide, vitamin A acid, azelaic acid, zinc, zinc sulfate, sulfur, salicylic acid, urea, fruit acids and mixtures thereof.

Component (b) is present in the composition of the present invention in a sufficient amount in order to be suitable for the prophylaxis and/or treatment of acne or for the cosmetic treatment of the skin. Typically, component (b) is present in the composition of the present invention in an amount of 0.01 to 20 wt %, preferably 0.05 to 15 wt %, more preferably 0.05 to 10 wt %.

The composition of the present invention is normally applied topically, i.e. on the parts of the skin which are to be conditioned or treated.

The composition of the present invention comprises preferably, in addition, at least one emulsifier. It is preferred that this emulsifier is selected from the group consisting of at least one ester, the carboxylic acid residue of which is derived from C₅-C₁₆ acids, and the hydroxy residue of which is derived from monomers, dimers or trimers of lactic acid or one of its salts or a polyglycerin of 2 to 10 molecules of glycerin whereby per mole of polyglycerin 1 to 3 mole of carboxylic acid are present.

The carboxylic acid residue of these esters is derived from the C₅-C₁₆ acids, preferably C₈₋₁₂ acids. The carbon chain of the carboxylic acid residue can be saturated or partially unsaturated. Preferred examples of the carboxylic acid residue include hexanoic acid, caprylic acid, capric acid, lauric acid, myristic acid, palmitic acid, stearic acid, behenic acid, oleic acid, linoleic acid and mixtures thereof, for example coconut fatty acid (the carboxylic acid residues of which are characterised by “cocoyl”) which is a mixture of the aforementioned fatty acids.

The hydroxy residue of the ester can be derived from monomers, dimers or trimers of lactic acid or one of its salts. Preferably a monomer or dimer of lactic acid is employed. It is furthermore preferred that the lactic acid is used in the form of its salt, i.e. as the lactate. Particularly preferred are alkali metal and alkaline earth metal salts, whereby sodium salts are particularly mentioned. Moreover, the hydroxy residue of the ester can be derived from a polyglycerin of 2 to 10 molecules of glycerin. In this case 1 to 3 moles of carboxylic acid are present per mole of polyglycerin. Particularly preferred 2 to 3 moles of carboxylic acid are present per mole of polyglycerin.

Examples of these emulsifiers which can be present in the composition of the present invention include dispersing auxiliaries as mentioned in DE-A-197 22 405, column 2, lines 38 to 56 as well as in the examples. Preferred are polyglycerin 10-tricaprylate, polyglycerin 10-trilaurate, polyglycerin 2-oleate, sodium lauryl lactate, sodium cocoyl lactate, capric/caprylic acid triglyceride and mixtures thereof. Particularly preferred are polyglycerin 2-oleate and sodium cocoyl lactate.

Further emulsifiers which may be present in the composition of the present invention include for example glycerin and sorbitan ester derivatives as well as cetearyl alcohol and ester derivatives thereof and mixtures of the substances. The glycerin, sorbitan and cetearyl ester derivatives are typically derived from esters whereby the carboxylic acid residues of which are derived from C₅₋₁₆ acids, the carbon chains of which are saturated or partially unsaturated. Particularly preferred of these are glycerin stearate, sorbitan stearate, sorbitan isostearate, sorbitan diisostearate, sorbitan dioleate, sorbitan distearate, sorbitan laurate, sorbitan palmitate, sorbitan sesquiisostearate, sorbitan sesquioleate, sorbitan triisostearate, sorbitan trioleate, sorbitan tristearate, cetearyl octanoate, cetearyl palmitate, cetearyl isononanoate and mixtures thereof.

The emulsifier is typically present in the composition of the present invention in an amount of 0.05 to 40 wt %, preferably 0.5 to 20 wt %, more preferably 1 to 10 wt %.

It is preferred that in addition at least one lipophilic solvent is present in order to improve the solubility of the active agents (components (a) and (b)) in the composition of the present invention. Typical lipophilic solvents suitable for a topic formulation include dimethicone, cyclomethicone, mineral oil, isostearyl isostearate, octyl palmitate, propylene glycol/dicaprate/dicaprylate, C₁₂₋₁₅ alkyl benzoate, octyl decanol, ether derivatives of cetyl alcohol such as Ceteth-1, Ceteth-2, Ceteth-3, Ceteth-4, Ceteth-5, Ceteth-6 and Ceteth-10, ethylbutylacetyl aminopropionate, ethanol, isopropanol, isopropyl myristate, and mixtures thereof. Of these ethylbutyl acetyl aminopropionate, ethanol, isopropanol, isopropyl myristate and mixtures thereof are particularly preferred. The addition of the lipophilic solvent can improve the solubility of the active agent so that the content of component (a) within the composition of the present invention can be increased. When adding the lipophilic solvent, the amount of component (a) in the composition of the present invention is preferably in the range of 0.01 to 30.wt %.

At least one lipophilic solvent is typically present in the composition of the present invention, in a sufficient amount in order to improve the solubility of the active agents, and it is preferably present in an amount of 0.1 to 20 wt %, more preferably 0.3 to 17 wt %.

Furthermore, it is preferred that at least one auxiliary, selected from antioxidants and UV filters, is contained in the composition of the present invention.

Apart from the known effects of the antioxidants and UV filters such as the protection from cell damage by radicals, or the protection from UV radiation and its damaging effects, the antioxidants and/or UV filters can further stabilize the active agents (components (a) and (b)). This has the effect of, e.g. an advantageous increase of the storage stability of the composition of the present invention.

The following antioxidants known from references in the respective field can be present in the compositions of the present invention, for example flavonides, coumaranones, amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoides, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthio uracil and other thiols (e.g. thioredoxin, glutathione, cysteine, cystine, cystamine as well as glycosyl, N-acetyl, methyl, ethyl, propyl, amyl, butyl, and lauryl, palmitoyl, oleyl, γ-linoleyl, cholesteryl and glyceryl esters of these) as well as salts of these, diaurylthiodipropionate, distearylthiodipropionate, thiodipropinoic acid and derivatives (esters, ethers, peptides, lipids, nucleotides, nucleosides and salts) of these as well as sulfoximine compounds (e.g., buthionine sulfoximines, homocysteine sulfoximine, buthionine sulfones, penta-, hexa-, heptathionine sulfoximine), furthermore (metal) chelating agents (e.g. α-hydroxy fatty acids, palmitic acid, phytic acid, lactoferrin), α-hydroxy acids (e.g. citric acid, lactic acid, malic acid), humic acid, gallic acid, gallic extracts, bilirubin, biliverdin, EDTA, EGTA and derivatives thereof, unsaturated fatty acids and derivatives thereof, vitamin C and derivatives (e.g. ascorbyl palmitate, magnesium ascorbyl phosphate, ascorbyl acetate) as well as coniferyl benzoate of the benzoic resin, rutinic acid and derivatives thereof, α-glycosyl rutin, ferulic acid, furfurylidene glucitol, carnosine, butylhydroxy toluene (BHT), butylhydroxy anisole, nordohydroguaiaretic acid, trihydroxybutyrophenon, uric acid and derivatives thereof, mannose and derivatives thereof, zinc and derivatives e.g. ZnO, ZnSO₄), selenium and derivatives thereof (e.g. selenium methionine), stilbenes and derivatives thereof (e.g. stilbene oxide, trans-stilbene oxide) and melatonin.

Mixtures of antioxidants are also suitable for use in the compositions of the present invention. Known and commercially available mixtures are, for example, mixtures containing, as active ingredients lecithin, L-(+)-ascorbyl palmitate and citric acid (e.g. Oxynex® AP), natural tocopherols, L-(+)-ascorbyl palmitate, L-(+)-ascorbic acid and citric acid (e.g. Oxynex® K LIQUID), tocopherol extracts from natural sources, L-(+)-ascorbyl palmitate, L-(+)-ascorbic acid and citric acid (e.g. Oxynex® L LIQUID), DL-α-tocopherol, L-(+)-ascorbyl palmitate, citric acid and lecithin (e.g. Oxynex® LM) or butylhydroxy toluene (BHT), L-(+)-ascorbyl palmitate and citric acid (e.g. Oxynex® 2004).

In a preferred embodiment of the invention the composition of the present invention contains as the antioxidant butylhydroxy toluene.

In another preferred embodiment the topical composition of the present invention contains as the, antioxidant one or more compounds, selected from flavonoides and/or coumaranones.

Flavonoides are understood as being the glycosides of flavanones, flavones, 3-hydroxyflavones (=flavanoles), aurones, isoflavones and rotenoides (Römpp Chemie Lexikon, Vol. 9, 1993). Within the scope of the present invention, however, also the aglycones, i.e. the sugar free components, and the derivatives of the flavonoides and the aglycones are embraced. Within the scope of the present invention coumaranones embrace also the derivatives thereof.

Preferred flavonoides are derived from flavanones, flavones, 3-hydroxyflavones, aurones and isoflavones, in particular flavanones, flavones, 3-hydroxyflavones and aurones.

Flavanones are characterised by the following basic structure:

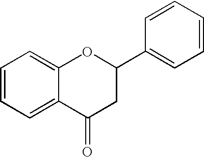

Flavones are characterised by the following basic structure:

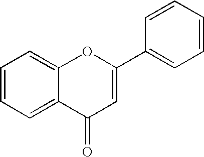

3-Hydroxyflavones (flavanoles) are characterised by the following basic structure:

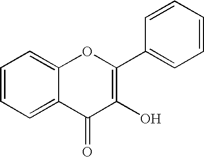

Isoflavones are characterised by the following basic structure.

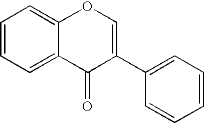

Aurones are characterised by the following basic structure:

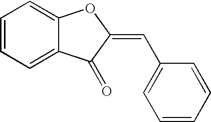

Coumaranones are characterised by the following basic structure:

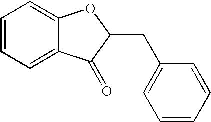

Preferably the flavonoides and coumaranones are selected from compounds of Formula (1):

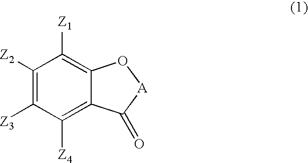

wherein

Z₁ to Z₄ each represent independently from each other H, OH, alkoxy, hydroxyalkoxy, mono- or oligoglycoside residues, whereby the alkoxy and hydroxyalkoxy groups can be branched or unbranched and may have 1 to 18 C-atoms and wherein on the hydroxy groups of the above mentioned residues, sulphate or phosphate may also be bonded,

A is selected from the group consisting of the substructures (1A), (1B) and (1C)

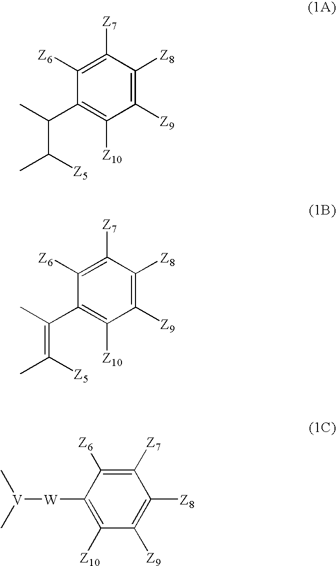

Z₅ represents H, OH or OR,

R represents a mono- or oligoglycoside residue,

Z₆ to Z₁₀ have the meaning of the residues Z₁ to Z₄ and

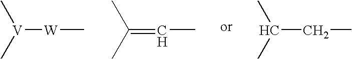

The alkoxy groups are preferably linear and have 1 to 12, preferably 1 to 8 C-atoms. These groups correspond, therefore, to Formula —O—(CH₂)ₘ—H, wherein m represents 1, 2, 3, 4, 5, 6, 7 or 8 and in particular 1 to 5.

The hydroxyalkoxy groups are preferably linear and have 2 to 12, preferably 2 to 8 C-atoms. These groups represent, therefore, Formula —O—(CH₂)ₙ—OH wherein n represents 2, 3, 4, 5, 6, 7 or 8, preferably 2 to 5 and in particular preferably 2.

The mono- and oligoglycoside residues are preferably made up from 1 to 3 glycoside units. Preferably these units are selected from the group of hexosyl residues, in particular rhamnosyl residues and glycosyl residues. However, other hexosyl residues, for example allosyl, altrosyl, galatosyl, gulosyl, idosyl, mannosyl and talosyl can be used advantageously under the circumstances. Moreover, it can be advantageous for the invention to use pentosyl residues.

In a preferred embodiment the substituents have the following meaning

Z₁ and Z₃ the meaning H,

Z₂ and Z₄ a different meaning than H, in particular they represent OH, methoxy, ethoxy or 2-hydroxyethoxy,

Z₅ the meaning H, OH or a glycoside residue made up of 1 to 3, preferably 1 or 2 glycoside units.

Z₆, Z₉ and Z₁₀ represent the meaning H, and

Z₇ and Z₈ a different meaning than H, in particular they represent OH, methoxy, ethoxy or 2-hydroxyethoxy.

In another preferred embodiment, in particular when the water solubility of the flavonides and coumaranones should be increased, a sulphate or phosphate group is bonded to the hydroxy groups. Suitable counter-ions are, for example, ions of alkali metals or alkaline earth metals, wherein these are, e.g. selected from sodium or potassium.

In another preferred embodiment, the flavonoides are selected from the following compounds: 4,6,3′,4′-tetrahydroxyaurone, quercetin; rutin, isoquercetin, anthocyanidin (cyanidin), eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin) as well as sulphates and phosphates thereof.

From the flavonoides, rutin and troxerutin are particularly preferred. Especially preferred is troxerutin.

From the coumaranones, 4,6,3′,4′-tetrahydroxybenzyl coumaranone-3 is preferred.

The antioxidants are typically incorporated into the compositions of the present invention in an amount of 0.001 to 5 wt %, preferably 0.5 to 5 wt %.

Suitable organic UV filters can be selected from all known UVA but also UVB filters known to the person skilled in the art. For both UV ranges there are reliable substances known from special literature, for example

Benzylidene camphor derivatives, such as

3-(4′-methylbenzylidene) dl-camphor (e.g. Eusolex 6300),

3-benzylidene camphor (e.g. Mexoryl® SD),

polymers of N-{(2 and 4)-[(2-oxoborn-3-ylidene)methyl]benzyl}acrylamide (e.g. Mexoryl® SW)

N,N,N-trimethyl-4-(2-oxoborn-3-ylidene methyl)anilinium methylsulphate (e.g. Mexoryl® SK) or

α-(2-oxoborn-3-ylidene)toluene 4-sulphonic acid (e.g. Mexoryl® SL).

Benzoyl or dibenzoyl methanes, such as

1-(4-tert-butylphenyl)-3-(4-methoxyphenyl) propane 1,3-dione (e.g. Eusolex® 9020) or

4-isopropyldibenzoyl methane (e.g. Eusolex® 8020)

Benzophenones, such as

2-hydroxy-4-methoxy benzophenone (e.g. Eusolex® 4360) or

2-hydroxy-4-methoxy benzophenone 5-sulphonic acid and its sodium salt (e.g. Uvinul® MS-40),

Methoxy cinnamates, such as

p-methoxy cinnamic acid 2-ethylhexylester (e.g. Eusolex® 2292),

p-methoxy cinnamic acid isopentylester, e.g. as a mixture of the isomers (e.g. Neo Heliopan® E 1000),

Salicylate derivatives, such as

2-ethylhexyl salicylate (e.g. Eusolex® OS),

4-isopropylbenzyl salicylate (e.g. Megasol®) or

3,3,5-trimethylcyclohexyl salicylate (e.g. Eusolex® HMS),

4-aminobenzoic acid and derivatives thereof, such as

4-aminobenzoic acid,

4-(dimethylamino)benzoic acid 2-ethylhexylester (e.g. Eusolex® 6007),

ethoxylated 4-aminobenzoic acid ethylester (e.g. Uvinul® P25),

and further substances such as

-2-cyano-3,3-diphenyl acrylic acid 2-ethylhexylester (e.g. Eusolex® OCR),

2-phenylbenzimidazole 5-sulphonic acid as well as its potassium, sodium and triethanol amine salts (e.g. Eusolex® 232),

3,3′-(1,4-phenylenedimethylene)-bis-(7,7-dimethyl-2-oxobicyclo[2.2.1]hept-1-yl methane sulphonic acid as well as its salts (e.g. Mexoryl® SX) and

2,4,6-trianilino-(p-carbo-2′-ethylhexyl-1′-oxy) 1,3,5-triazine (e.g. Uvinul® T 150).

These organic UV filters are typically incorporated into the compositions of the present invention in an amount of 0.5 to 10 wt %, preferably 1 to 8 wt %.

Further suitable organic UV filters are, for example,

2-(2H-benzotriazol-2-yl)-4-methyl-6-(2-methyl-3-(1,3,3,3-tetramethyl-1-(trimethylsilyloxy)disiloxanyl)propyl) phenol (e.g. Silatrizole®),

4,4′-[(6-[4-((1,1-dimethylethyl)aminocarbonyl)phenylamino]1, 3,5-triazin-2, 3-diyl)diamino]bis-benzoic acid 2-ethylhexylester (e.g. Uvasorb® HEB),

α-(trimethylsilyl)-ω[trimethylsilyl)oxy]poly[oxy(dimethyl] [and approx. 6% methyl[2-[p-[2,2-bis-(ethoxycarbonyl]vinyl]phenoxy]-1-methylene ethyl] and approx. 1.5% methyl[3-[p-[2,2-bis-(ethoxycarbonyl)vinyl)phenoxy) propenyl) and 0.1 to 0.4% (methylhydrogen]silylene]] (n≈60) (e.g. Parsol® SLX),

2,2′-methylene-bis-(6-(2H-benzotriazol-2-yl) 4-(1,1,3,3-tetramethylbutyl) phenol (e.g. Tinosorb® M),

2,2′-(1,4-phenylene)-bis-1H-benzimidazole 4,6-disulphonic acid monosodium salt,

2,2′-(1,4-phenylene)-bis-1H-benzimidazole 5-disulphonic acid monosodium salt,

2,2′-(1,4-phenylene)-bis-1H-benzimidazole 5-disulphonic acid monopotassium salt, and

2,4-bis-[4-(2-ethyl-hexyloxy)-2-hydroxyl]-phenyl 6-(4-methoxyphenyl) 1,3,5-triazine (e.g. Tinosorb® S).

These organic filters are typically incorporated into the compositions of the present invention in an amount of 0.5 to 20 wt %, preferably 1 to 15 wt %.

Inorganic UV filters can be selected from the group of titanium dioxides, e.g. coated titanium dioxide (e.g. Eusolex® T-2000 or Eusolex® I-Aqua), zinc oxide (e.g. Sachtotec®), iron oxides or also cerium oxides. These inorganic UV filters are typically incorporated into the compositions of the present invention in an amount of 0.5 to 20 wt %, preferably 2 to 10 wt %.

Preferred UV filters are zinc oxide, titanium dioxide, 3-(4′-methylbenzylidene) d-camphor, 1-(4tert-butylphenyl) 3-(4-methoxyphenyl)propan-1,3-dione, 4-isopropyl dibenzoyl methane, 2-hydroxy-4-methoxybenzophenone, 4-methoxy cinnamic acid 2-ethylhexyl ester, 3,3,5-trimethylcyclohexyl salicylate, 4-(dimethylamino)benzoic acid 2-ethylhexyl ester, 2-cyano-3,3-diphenyl acrylic acid 2-ethylhexyl ester, 2-phenyl benzimidazole-5-sulphonic acid, as well as their potassium, sodium and triethanol amino salts.

Especially preferred UV filters are zinc oxide and titanium dioxide.

From the compositions of the present invention containing titanium dioxide, those are preferred which contain apart from titanium dioxide, in addition, one or more further UV filters selected from 3-(4′-methylbenzylidene) dl-camphor, 1-(4-tert-butylphenyl)-3-(4-methoxyphenyl)propan-1,3-dione, 4-isopropyl dibenzoyl methane, 2-hydroxy-4-methoxybenzophenone, p-methoxycinnamic acid 2-ethylhexyl ester, 3,3,5-trimethylcyclohexyl salicylate, 4-(dimethylamino)benzoic acid 2-ethylhexyl ester, 2-cyano-3,3-diphenyl acrylic acid 2-ethylhexyl ester, 2-phenyl benzimidazole 5-sulphonic acid, as well as their potassium, sodium and triethanol amine salts.

In order to improve the protection of the skin and an immuno suppression of the skin, the combination of aryl oximes with ectoin and ectoin derivatives is particularly effective.

From these compositions, those are particularly preferred, which include apart from titanium *dioxide, in addition, the UV filters 2-hydroxy-4-methoxy benzophenone and/or p-methoxy cinnamic acid 2-ethylhexyl ester.

Depending on its use, the composition of the present invention can contain under the circumstances further auxiliaries and/or carrier agents such as carriers, preservatives, stabilisers, solvents, vitamins, colouring agents, agents to improve the odour, film-forming agents, thickening agents and moisturisers.

Forms of using the composition of the present invention include, for example, solutions, suspensions, emulsions, pastes, ointments, gels, creams, lotions, powders, soaps, surfactant containing cleaning preparations, oils, sticks and sprays.

Ointments, pastes, creams and gels may contain the usual carriers, e.g. animal and vegetable fats, waxes, paraffins, starch, tragacanth, cellulose derivatives, polyethylene glycols, silicones, bentonites, silica, talcum and zinc oxide, xanthane gum, glycerin, carboxypolymethylene or mixtures of these compounds.

Powders and sprays may contain the usual carriers, e.g. lactose, talcum, silica, aluminium hydroxide, calcium silicate and polyamide powder or mixtures of these compounds. Sprays may additionally contain the usual propellants, e.g. chlorofluoro carbons, propane/butane or dimethyl ether.

Solvents and emulsions may contain the usual carriers, such as solvents, solubilising agents and emulsifiers, e.g. water, ethanol, isopropanol, ethyl carbonate, ethyl acetate, benzyl alcohol, benzyl benzoate, propylene glycol, 1,3-butyl glycol, oils, in particular, cottonseed oil, peanut oil, maizegerm oil, olive oil, castor oil and sesame oil, esters of glycerin fatty acids, polyethylene glycols, xanthane gum, glycerin, carboxypolymethylene and fatty acid esters of sorbitan or mixtures of these compounds.

Suspensions may contain the usual carriers, such as liquid diluents, e.g. water, ethanol or propylene glycol, suspending agents, e.g. ethoxylated isostearyl alcohols, polyoxyethylene sorbitan esters and polyoxyethyl sorbitan esters, microcrystalline cellulose, aluminium metahydroxide, bentonite, agar-agar gum and tragacanth, xanthane gum, glycerin, carboxypolymethylene or mixtures of these compounds.

Soaps may contain the usual carriers such as alkali metal salts of fatty acids, salts of fatty acid semi-esters, fatty acid protein hydrolysates, isothionates, lanolin, fatty alcohol, vegetable oils, vegetable extracts, glycerin, sugar or mixtures of these compounds.

Surfactant containing cleaning products may contain the usual carriers such as salts of fatty alcohol sulphates, fatty alcohol ether sulphates, sulphosuccinic acid semi-esters, fatty acid protein hydrolysates, isothionates, imidazolinium derivatives, methyl taurates, sarcosinates, fatty acid amide ether sulphates, alkyl amidobetaines, fatty alcohols, fatty acid glycerides, fatty acid diethanol amides, vegetable and synthetic oils, lanolin derivatives, ethoxylated glycerin fatty acid esters or mixtures of these compounds.

Facial and body oils may contain the usual carriers such as synthetic oils, such as fatty acid esters, fatty alcohols, silicone oils, natural oils such as vegetable oils and oily plant extracts, paraffin oils, lanolin oils or mixtures of these compounds.

The composition of the present invention may be prepared according to usual processes. Different process techniques can be employed in order to mix the components of the composition according to the present invention as closely as possible. Depending on the required mixing intensity, one or more processes of blending compounds are used which may run subsequently or in a parallel order.

In chemical process engineering, one understands by the term “mixing” basic operations which lead to a homogenisation of compounds to the greatest possible extent. Compound streams should be combined in such a manner so that in partial volumes of the formed mixture a composition of the single components which is as homogeneous as possible is obtained.

A special form of mixing is termed homogenisation. This is a mixing of phases which are normally immiscible with each other. Thus, homogenisation is, therefore, a change of the distribution and the particle size of the inner phase of emulsions and suspensions, so that a homogeneous system is formed when observed under the microscope, and the distributed phase will not be deposited or does not form a cream without the action of external forces.

Dispersion is understood as a mixing of a compound system consisting of two or more phases in which one compound (dispersed phase) is distributed (dispersed) most finely in another one (dispersing agent). The particles of the dispersed phase as well as the dispersing agent may be solid, liquid or gaseous. Examples of dispersions are aerosols, emulsions, suspensions and colloids.

Another usual type of mixing in the preparation of cosmetics is emulsification. This is the mixing of two liquids which are immiscible or only slightly miscible with each other, whereby one of the liquids is finely distributed in the other. The outer phase is described as the continuous phase or the dispersing agent, the liquid distributed therein is described as the inner, discontinuous or dispersed phase. Cosmetic emulsions consist in most cases of an aqueous polar phase and an non-polar oil phase.

Suspending, on the other hand, is the distribution of very small but not molecular particles of a solid compound or a liquid. Just like emulsions, suspensions are often optically opaque and have the tendency to deposit under the influence of gravity. The mixing processes mentioned above are suitable for the preparation of the composition of the present invention. It is particularly preferred that the composition of the present invention is prepared by homogenisation, dispersion or emulsification.

The present invention is described in more detail by the following examples.

### EXAMPLE 1

O/W-Emulsion

| | | |
|---|---|---|
| propylene glycol | 4.0 | wt % |
| glycerin monostearate | 4.0 | wt % |
| isopropyl myristate | 4.0 | wt % |
| stearic acid | 4.0 | wt % |
| propylene glycol monostearate | 4.0 | wt % |
| 2-hydroxy-5-methyl laurophenone oxime | 1.0 | wt % |
| vitamin A acid | 0.05 | wt % |
| distilled water to make | 100 | wt % |

These components were emulsified to prepare a cream which can be used for acne therapy.

### EXAMPLE 2

O/W-Emulsion

| | | |
|---|---|---|
| propylene glycol | 4.0 | wt % |
| glycerin monostearate | 4.0 | wt % |
| isopropyl myristate | 4.0 | wt % |
| stearic acid | 4.0 | wt % |
| propylene glycol monostearate | 4.0 | wt % |
| 2-hydroxy-5-methyl laurophenone oxime | 1.0 | wt % |
| benzoyl peroxide | 5.0 | wt % |
| distilled water to make | 100 | wt % |

These components were emulsified to prepare a lotion suitable for acne therapy.

1. A composition, comprising
(a) at least one aryl oxime of Formula (I) and
(b) at least one active agent for the treatment of acne 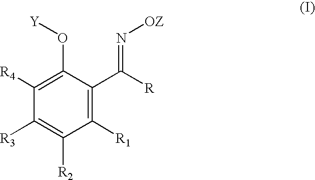
wherein:
Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;
R represents C₁₋₁₈ alkyl, C₂₋₁₈ alkenyl, C₃₋₈ cycloalkyl, aryl, aralkyl, heteroaryl, heteroaralkyl or condensed systems;
R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl: amine or sulfonyl.

2. Composition according to claim 1, wherein the component (a) is present in the composition in an amount of 0.02 to 2 wt %.

3. Composition according to claim 1 or 2, wherein the component (b) is selected from the group consisting of chemotherapeutics, antibiotics, antiandrogens, retinoids, benzoyl peroxide, vitamin A acid, azelaic acid, zinc, zinc sulphate, sulfur, salicylic acid, urea, fruit acid and mixtures thereof.

4. Composition according to one of claims 1 to 3 wherein the component (b) is present in the composition in an amount of 0.01 to 20 wt %.

5. Composition according to one of claims 1 to 3 wherein the composition comprises furthermore at least one carrier or an auxiliary selected from at least one of the following:
an emulsifier, a lipophilic solvent, an antioxidant, a UV filter and mixtures thereof.

6. Use of the composition according to one of claims 1 to 4 for the prophylaxis and/or treatment of acne.

7. Use of the composition according to one of claims 1 to 4 for the cosmetic care of the skin.

8. Use of at least one 1-(2-hydroxyaryl)-alkan-1-one oxime of Formula (1) 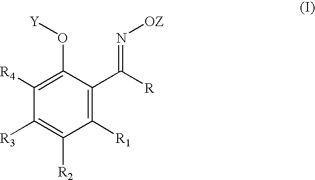
wherein:
Y, Z represent independently from each other H, C₁₋₁₈ alkyl, C₂₋₁₈ alkenyl, C₂₋₁₈ carboxy alkyl, C₃₋₁₈ carboxy alkenyl or C₂₋₁₈ alkanoyl;
R represents C₁₋₁₈ alkyl, C₂₋₁₈ alkenyl, C₃₋₈ cycloalkyl, aryl, aralkyl, heteroaryl, heteroaralkyl or condensed systems;
R₁, R₂, R₃ and R₄ represent independently from each other H, C₁₋₁₂ alkyl, C₂₋₁₂ alkenyl, C₁₋₁₂ alkoxy, C₃₋₈ cycloalkoxy, aryl, aryloxy, aralkyl, heteroaryl, heteroaralkyl, carboxy, hydroxy, chlorine, dialkyl amine or sulfonyl,
for the prophylaxis and/or treatment of skin reddening and/or inflammations caused by acne and/or acne therapy.
